(19) United States
(12) Patent Application Publication
(10) Pub. No.: US 2004/0009238 A1
(43) Pub. Date: Jan. 15, 2004

(54) Exogenenous nitric oxide gas (gNO) therapy in wound healing

(76) Inventors: Chris Miller, North Vancouver (CA); Abdi Ghaffari, Edmonton (CA); Ali Ardakani, North Vancouver (CA)

Correspondence Address: Kevin D. McCarthy, Roach Brown McCarthy & Gruber, P.C., 1620 Liberty Building, 420 Main Street, Buffalo, NY, 14202, US

(21) Appl. No.: 10/615,546

(22) Filed: Jul. 8, 2003

Related U.S. Application Data
(60) Provisional application No. 60/431,876, filed on Dec. 9, 2002.
(60) Provisional application No. 60/394,690, filed on Jul. 9, 2002.
(60) Provisional application No. 60/409,400, filed on Sep. 10, 2002.

(51) Int. Cl. A61K 33/00

(52) U.S. Cl. 424/718

(57) ABSTRACT

The present invention provides a method and device for exposing injured mammalian tissues, in a non-abrasive manner, to an effective amount of exogenous gaseous nitric oxide (gNO) in order to promote healing by reducing the size, duration and severity of wounds as well as controlling the infection by reducing number of pathogens at the site and the surrounding area.

### CLAIM OF PRIORITY

This application claims priority to U.S. provisional Patent applications Nos. 60/431,876 (filed Dec. 9, 2002), 60/394690 (filed Jul. 9, 2002), and 60/409400 (filed Sep. 10, 2002).

### FIELD OF THE INVENTION

This invention pertains to a method and device for delivery of gaseous nitric oxide (gNO). The NO is directed to a wound on a mammal to promote the healing of the wound.

### BACKGROUND OF THE INVENTION

Nitric oxide (NO) is an intensely studied molecule in medical science. It is a short-lived free radical. It is also highly reactive and locally diffusible because of its small molecular size and unpaired electron. Since its discovery as an endothelium derived relaxing factor in 1987, it has become evident that NO is a widely distributed multi-functional intra- and inter-cellular messenger. NO is formed from a terminal nitrogen atom of arginine through an oxidation process with molecular oxygen. It is understood that certain enzymes, referred to as nitric oxide synthases (NOS), are responsible for that oxidation process.

NO has also been shown to have a direct or an indirect role in pathophysiology of numerous bodily functions both in human and mammals. Some of these bodily functions and disorders include but not limited to (1) blood flow and pressure in body circulatory system, (2) pulmonary hypertension, (3) asthma, (4) inflammatory response, (5) infection, (6) cancer, (7) angiogenesis, (8) neurotransmission in nervous system, (9) diabetes, and (10) sexual dysfunction such as penile erection. Over the past several years, NO has also been noted to play an important role in wound healing.

Conventionally wounds heal through a three step process. The first step is called an initial inflammatory phase. This phase is defined by platelet aggregation, degranulation, and phagocytosis.

The second step is referred to as the proliferative phase. This phase is characterized by an expansion of reparative cells. The reparative cells include fibroblasts. Fibroblasts are a major synthetic element in a wound, and are responsible for production and reorganization of structural proteins (such as collagen) required for tissue repair. Endothelial migration and angiogenesis also initiate in this stage.

The third and last step is called the maturation phase. This phase is the longest stage in the wound healing process. In this phase, newly deposited collagen (from fibroblasts) and an extracellular matrix are reorganized and result in increasing wound strength and eventually in mature scar formation.

NO is both directly and indirectly, as a regulator, involved in each of these physiological steps. In fact, many wound resident cells have the ability to synthesize or affect the synthesis of NO. Examples of wound resident cells include and are not limited to macrophages, neutrophiles, endothelial cells, vascular smooth muscle cells, keratinocytes, lymphocytes, and fibroblasts.

Lack of NO and arginine in mammals result in a decrease in (a) NO metabolism, (b) wound breaking strength, (c) collagen synthesis, (d) epithelialisation, and (e) wound contraction. In complementary studies that used chemical NO donors and arginine rich diet, the results point toward an increase in all of above factors, which result in the promotion and acceleration of the wound healing.

NO is also a known factor in promoting angiogenesis (development and rearrangement of new blood vessels within an injured tissue), increasing circulation to injured site, stimulating collagen synthesis in fibroblast, and mediating growth factor release. There are many situations a wound's healing response is delayed or inhibited in patients with systemic diseases. Systemic diseases include and are not limited to liver failure, renal impairment, diabetes, peripheral vascular disease, or in patients taking drugs like corticosteroids or immunosuppressive agents that inhibit healing, or prolonged process of healing in elderly. In all these cases, additional exogenous NO gas enhance the healing process.

Keloids and hypertrophic scars are examples of scarring pathology that is characterized by excess collagen deposition during process of wound healing. The exact mechanism of this disorder is not well understood, but it is shown that NOS expression and NO production are significantly reduced in fibroblasts derived from hypertrophic scars. By maintaining high levels of NO in these wounds, exogenous gNO can offer a potential treatment.

There are many situations in which the healing response in a wound is delayed or inhibited in patients with systemic diseases. In all these cases, additional exogenous gNO can potentially enhance or accelerate the wound healing process. One of these areas that gNO can have vast therapeutic impact is patients with diabetes dealing with complicated non-healing wounds. As mentioned above, a systemic deficiency of endothelial derived NO has been observed in diabetics, suggesting that NO plays a fundamental role in the pathogenesis of chronic, non-healing wounds. Diabetes affects an estimated 15 million people in the US alone.

In flap and micro-surgery reperfusion to ischemic tissue and organs is a critical criterion in survival of the tissue. Therefore administration of exogenous gNO, due to its vasodilatory and angiogenesis effects, can potentially maintain the vascular tone and protect the skin flap.

Secondary infection in chronic and open wounds can seriously slow down or complicate the process of healing. NO antimicrobial has been well documented in literature and supported by applicant's in vitro and animal studies using gNO. Nitric oxide has clearly shown bactericidal and/or bacteristatic effects on at least two of the most common pathogens in chronic wounds, namely pseudomonas auroginosa and staphylococcus aureus.

In PCT International Application number PCT/CA99/01123, the assignee of this application disclosed a method and device for treatment of respiratory infections by NO gas inhalation. This property of NO is critical in controlling an infection and giving the immune system a chance to fight and clear out the pathogens.

In U.S. Pat. No. 6,432,077, Stenzler discloses “device and method for treatment of surface infections with nitric oxide.” Stenzler also discloses that “while NO has shown promise with respect to certain medical applications, delivery methods and devices must cope with certain problems inherent with gaseous NO delivery. First, exposure to high concentrations of NO is toxic, especially exposure to NO in concentrations over 1000 ppm. Even lower levels of NO, however, can be harmful if the time of exposure is relatively high. For example, the Occupational Safety and Health Administration (OSHA) has set exposure limits for NO inhalation in the workplace at 25 ppm time-weighted average for eight (8) hours.” In other words, Stenzler promoted that exposing injured skin to NO in excess of 25 ppm time-weighted average for eight hours is deleterious to the skin. We have found that such limited exposure does not effectively promote the wound healing process. In addition, our in vitro and in vivo studies show no toxic effect to skin cells (such as fibroblast) following continuous exposure to 400 ppm gNO. In order to comply with OSHA guideline for inhalation of NO by workers or patients, commercially available filters (e.g. No. 67-35-813 Drager Industrial Ltd) can be used at the exhaust valve to scavenge NO and nitrogen dioxide (NO₂).

In view of the above, there is a need in the art for therapies that improve and accelerate the healing process in wounds through a temporally regulated manner, with specific attention to chronic wounds such as non-healing diabetic foot ulcers, 3^{rd} degree burns, venous and pressure ulcers, and hypertrophic scarring and keloids.

### SUMMARY OF THE INVENTION

The present invention provides a method and device for exposing injured mammalian tissues, in a non-abrasive manner, to an effective amount of exogenous gaseous nitric oxide (gNO) in order to promote healing by supporting skin cell growth, angiogenesis and tissue perfusion, and reducing the size, duration and severity of wounds.

### BRIEF DESCRIPTION OF THE DRAWINGS

FIG. 1 illustrates a cross sectional diagram of one embodiment of the gNO delivery wound cover device.

FIG. 2 illustrates an alternate embodiment of the wound cover.

FIG. 3 illustrates one embodiment of an inflatable bathing unit for delivery of gNO to wound covers in FIGS. 1 and 2.

FIG. 4 illustrates schematics of gNO chamber designed to carry out in vitro studies for exposing human cultured cells as well as microorganisms to various concentrations of gNO, under optimal growth conditions.

FIG. 5 illustrates morphology of fibroblast cells exposed inside gNO chamber to less than 200 ppm versus control group inside conventional tissue culture incubator.

FIG. 6 illustrates in vitro growth of fibroblast cell following exposed to 20 and 200 ppm in comparison with control.

FIG. 7 illustrates cell attachment capacity of human fibroblasts following exposure to 160 ppm gNO.

FIG. 8 illustrates wound bacterial content following topical application of 200 ppm gNO in a full thickness infected wound model in rabbits.

FIG. 9 shows wound bacterial content following topical application of 400 ppm gNO in a full thickness infected wound model in rabbits.

FIG. 10 shows rabbit blood serum NOx (NO₂ & NO₃) levels following topical application of 400 ppm gNO.

FIG. 11 illustrates rabbit blood methemoglobin levels following topical application of 400 ppm gNO on a full thickness infected wound model.

FIG. 12 shows mRNA expression for collagen and collagenase following exposure to 200 ppm gNO for 24 and 48 hours.

FIG. 13 illustrates histology analysis of full thickness infected wound exposed to 200 ppm gNO for 24 hours.

### DETAILED DESCRIPTION OF THE PREFERRED EMBODIMENTS

It has been found that exogenous nitric oxide gas acts as an initiator of wound healing in mammals. First aspect of the invention encompasses promoting and accelerating the process of wound healing by a topical application of medical grade exogenous nitric oxide gas, in a concentration dependent manner, to an injured tissue (e.g. skin, bone, tendon, ligament, cornea, or other tissues) in a mammal.

In another aspect, the invention provides a method for increasing local blood flow at the wound site or in the immediate vicinity thereof, through an increase in local concentration of nitric oxide. Exogenous nitric oxide gas is a potent and effective vasodilator that can accelerate tissue perfusion and maintain vascular tone at the site of injury. Through this action, it will bring more nutrient, oxygen, inflammatory and healing factors to the injured tissue resulting in faster healing and closure.

In another aspect, the invention encompasses a device for localized delivery of an effective amount of exogenous gNO to the wound site by utilizing a specialized wound cover that covers the surface of the wound, and isolating it from the external environment.

In yet another aspect, it is also the aim of this invention to prevent further infection (secondary infection) by isolating the wound area from external environment using a transparent wound cover device for the delivery of the gas. This also prevents further wound dehydration.

In yet another aspect, gNO therapy is administered topically at the site of the wound immediately post trauma, or applied during a surgery procedure. In case of chronic or non-healing wounds, therapy illustrated in this invention can be administered continuously for as long as 4 weeks.

Briefly stated, the present invention provides a non-abrasive method that will accelerate and improve wound healing, particularly in situations where complicating factors are present such as, and not limited to, diabetic conditions, foot ulcers, venous and pressure ulcers, post surgery hospital acquired infectious wounds, non healing wounds in elderly and/or immunocompromised, keloids, hypertrophic scarring, burns, and skin flaps. In particular, it is believed that the present invention promotes wound strength and healing by activating the production of fibroblasts, stimulating synthesis of collagen, and initiating angiogenesis. It is also the aim of this invention to increase the local blood flow to the site of injury and by doing so, bring more nutrients and oxygen to the wound.

The present invention describes a new method and device for improving and accelerating the healing process of wounds in mammals, with particular attention to non healing and chronic wounds such as diabetic conditions, foot ulcer, venous and pressure ulcers, wounds in elderly and immunocompromised, and 3^{rd} degree burns. A preferred embodiment of the present invention delivers an external source of nitric oxide gas at an effective dose range and for optimal duration to the injured tissue such as, but not limited to, skin, bone, tendon, ligament, and cornea.

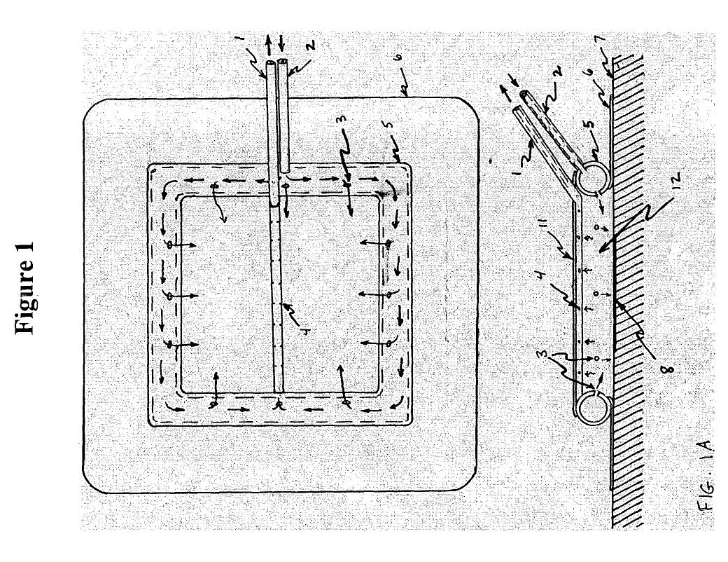

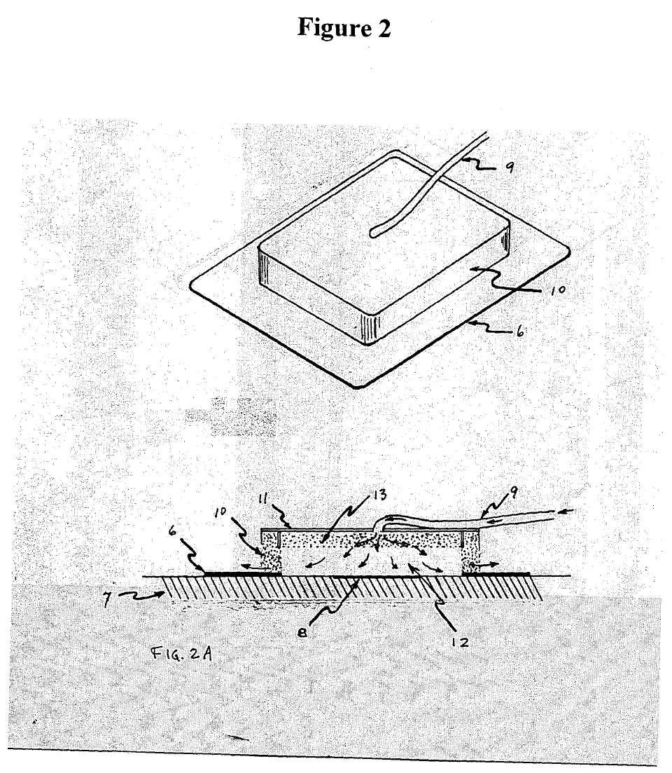

Methods and apparatus for delivery of exogenous nitric oxide gas from an external source to the wound cover (FIGS. 1 and 2) are disclosed herein and other methods have been disclosed in U.S. Pat. No. 6,432,077, and commonly assigned PCT International Application number PCT/CA99/01123. Applying gNO promotes wound healing in bodily injuries or lesions, in mammals. The injuries or lesions can be caused by physical means such as mechanical, chemical, viral, bacterial, or thermal means, which disrupt the normal continuity of structures.

The proposed therapy of this application facilitates the process of wound healing through suppression of inflammation, and the stimulation of cellular viability and proliferation which will lead into an increase in wound breaking strength, collagen synthesis, epithelialisation, and wound contraction.

In order to be effective in enhancing wound healing, the local concentration of nitric oxide in the injured tissue is increased through continuous or intermittent exposure to an effective dose of gNO. Dose level and duration of exposure will vary depending on nature and extent of the injury and will be assigned by those skilled in the art. Therapeutic dose of gNO will vary from 20 parts per million (ppm) to 1,000 ppm. The time for treatment will vary from 1 to 8 hours intermittent exposures daily or continuous exposure ranging from 1 to 31 days.

gNO therapy is administered topically at the different site of the wound immediately post trauma, or even applied during a surgical procedure. In case of chronic wounds, e.g., diabetic conditions, foot ulcer, tennis elbow, jumper's knee, or in non-healing wounds, this therapy will be prolonged by those skilled in the art until desired healing effect has been obtained. It is hereby suggested that prolonged exposure, in excess of 8 hours, and from 1 to 4 weeks, to an effective dose of exogenous gNO has a therapeutic effect in treating conditions dealings with complicated or non-healing wounds.

A gNO wound cover is preferred over other types of wound covers (e.g. bandages or dressing) because the same gNO wound cover can be repeatedly connected to a gNO source 1 at a desired exposure interval and temperature and does not need to be changed or removed following gNO therapy. In contrast, repeated application and removal of bandages and/or dressings will increase the probability of microbial infection. Additionally, airtight bandages and/or dressings prohibit moisture from escaping. Thereby bacteria, present at the time of procedure, to flourishes under the bandage.

FIG. 1 illustrates a cross sectional diagram of one embodiment of the non-contact gNO wound cover device. The wound cover consists of a sealing lip 6, which attaches to the uninjured skin 7, surrounding the wound 8 completely. The rigid ring 5 forms the wall and a barrier layer 11 attached to the top of ring 5 forms the remainder of the cover thus providing an enclosed treatment volume 12 over the wound. The wound cover is preferably constructed of a clear plastic such as, but not limited to, polyvinyl chloride which is stiff but which may conform to the shape of the surface of the body of the patient. Gases such as gNO, air or other gaseous mixtures are introduced into the treatment volume area 12 by a supply line 2 which flows into a ring 5. Orifices 3 on the inside wall of the ring 5 allow the gas to enter the treatment volume area 12. The spent gas is exhausted through the orifices 4 of an exhaust tube 2. A sealing lip 6 attaches to the skin 7 by an adhesive.

FIG. 2 illustrates an alternate embodiment of the device shown in FIG. 1. In this embodiment of the device, the sealing lip 6 attaches to the uninjured skin 7 by means of an adhesive, surrounding the wound 8 completely. The sides of the ring 10 are in this embodiment constructed of gas permeable but stiff foam which is flexible enough to conform to the patient's body. A barrier layer 11 covers the top of the device. Below and attached to the barrier layer 11 is a layer of gas and liquid permeable porous foam 13 through which the gases introduced through delivery line 9 must pass. The purpose of the porous foam layer 13 diffuses the gas flow. Also through this porous foam layer 13 additional medications or wound healing agents (gas or liquid) may be administered through deliver line 9 in order to alter the environment of treatment volume area 12. The treatment gases introduced into the treatment volume area 12 exhaust through the permeable walls 10 of the device.

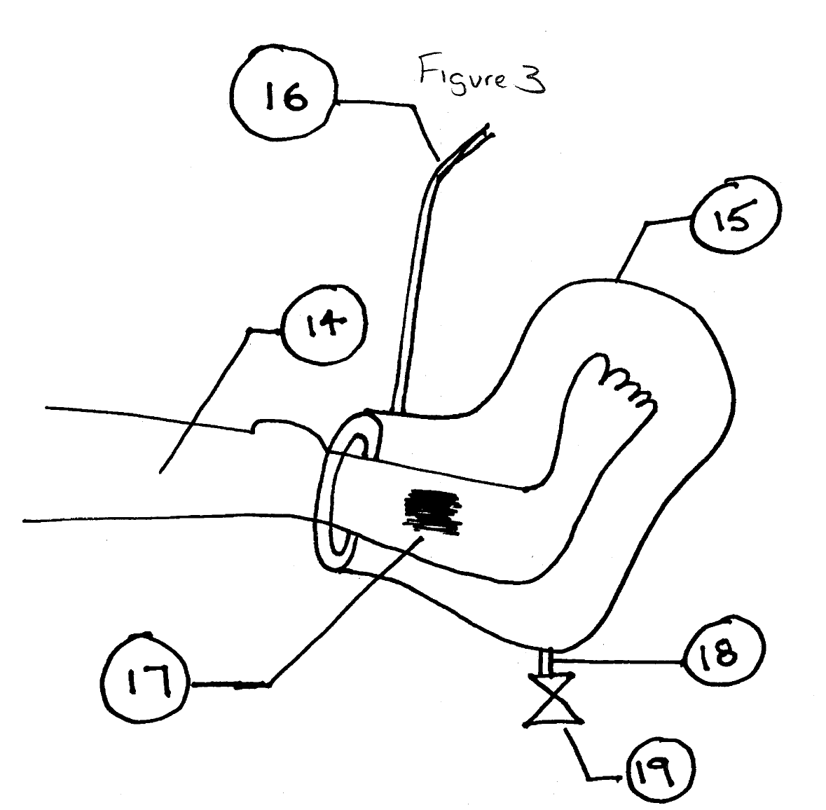

FIG. 3 shows an inflatable bathing unit 15 that can be used on its own or in combination with wound cover shown in FIGS. 1 and 2 when a higher therapeutic dose of gNO is being administrated and the risk of prolonged exposure to nitrogen dioxide (NO₂) should be avoided. The bathing unit 15 can take the shape of a boot that is placed over the patient's foot 14. The inlet line 16 can be connected to a gNO delivery device (e.g. AeroNOx, Pulmonox Medical Inc) titrating the exact amount of gNO desired in therapy. The delivery line 18 in the bathing unit can be connected to delivery line 9 of the wound cover and excess gas mixture can exit the unit through the one way exhaust valve 18. In order to scavenge excess gNO and nitrogen dioxide (NO₂), a specialized filter 19 is attached to the exhaust valve 18 (e.g. No. 67-35-813, Drager Industrial Ltd).

It is understood by those skilled in the art that although the embodiments of the devices illustrated in FIG. 1 and FIG. 2 are rectangular in shape, the device could be supplied in a variety of shapes and sizes. Additionally the size of the required treatment volume and the size of the wound will determine the size of the sealing lip 6 and the size and thickness of the ring 5 and barrier layer 11 to prevent contact with the wound. It would also be understood by those skilled in the art that other embodiments of the device can also be produced to include alternative methods of dispensing of the spent gases such as through filters in the barrier layer, or construction of the device as a number of parts that could be assembled as required to form any desired shape or size without departing from the scope of the invention.

Devices in FIGS. 1 and 2 when used with gases such as gNO and air mixtures, complete exchange of the treatment volume at a rate of three times per minute produced negligible formation of NO₂ with treatment volumes of 200 milliliters. The size of the device and treatment volume will determine the flow of gas necessary to maintain minimal formation of NO₂.

In another aspect of the invention, gNO can be delivered locally as a spray stored in small pressurized cylinders at a preset concentration immediately following a trauma, where gNO delivery system and wound cover are not accessible.

Due to the active role of nitric oxide in various physiological processes, for optimal use of the present invention, gNO should be delivered locally, i.e., take within or in the immediate vicinity of an injured tissue. Nitric oxide is highly reactive with air oxygen and iron molecule in heme moiety of hemoglobin leading to production of NO₂ and methemoglobin, respectively. Levels of these two by-products will be monitored closely in sampled air from the wound cover by a chemiluminescence analyzer (e.g. AeroNOx, PulmoNOx Medical Inc.) and blood for the duration of gNO therapy.

Particular pretreatment methods can be particularly advantageous prior to or in conjunction with gNO therapy. For example, gNO therapy can be preceded by mechanically scraping the surface of the wound in order to remove necrotic tissue and debris from the wound surface and increase the penetration power of gNO molecule into the injured area. The invention may also be used in combination with various agents including antibiotics, anesthetics, analgesics, anti-inflammatory agents such as corticosteroids and nonsteroidal anti-inflammatory agents, antiviral agents, vasodilators or vaso-constrictors, antihistamines, other hormones such as estrogens, progesterone, androgens, antiseborretic agents, other cardiovascular agents, mast cell stabilizers, scabicides or pediculicides, keratolytics, lubricants, narcotics, shampoos, burn preparations, cleaning agents, photosensitizing agents, wet dressings and other wound care products in order to further enhance the healing process. Other agents may be employed in combination with gNO therapy to indirectly enhance the local amount of nitric oxide, e.g., by enhancing absorption or prolonging therapeutic effects (such as phosphodiesterase inhibitors), and/or to enhance the activity of NO synthase, or to protect NO from degradation.

The types of tissue that may be treated using methods in the present invention include without limitation human and other mammalian muscle, tendon, ligament, skin, mucosa, bone, cartilage, and cornea. The tissue may be damaged by surgical incisions, trauma (mechanical, chemical, viral, bacterial, or thermal in nature), or other endogenous pathological processes. Healing may be impaired as a result of systemic diseases.

Foot ulcers are a potentially serious complication in diabetics as the healing process is inhibited by a decrease in wound capillaries, fibroblasts, and collagen at the wound site and by immune system's inability to fight infection. The present invention elevates the synthesis of collagen through production of wound fibroblast at the injured site. Through vasodilatory action of gNO, the present invention increases the local blood flow and perfusion to the extremities where the wound is located.

Experiments

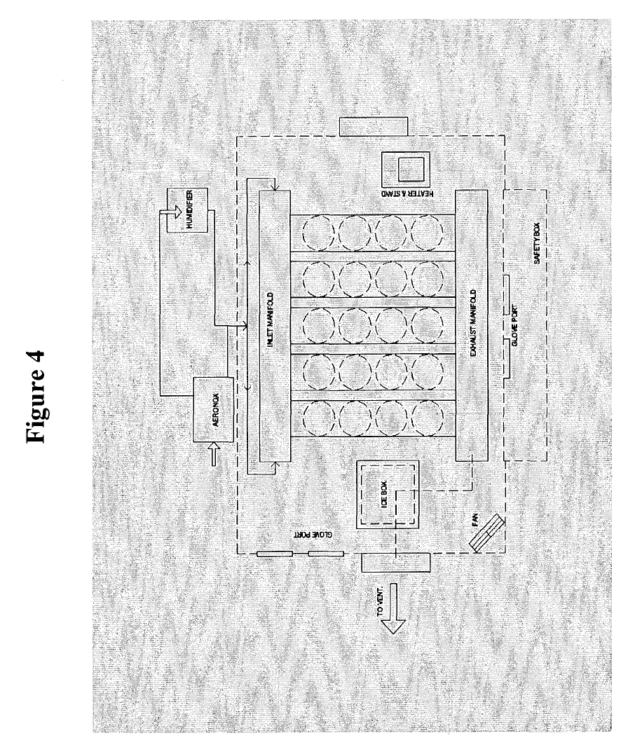

Device: FIG. 4 shows a specialized gNO incubation chamber designed for conducting in vitro studies on mammalian cell cultures as well as bacterial cells under optimal growth conditions to study the effect of gNO exposure on mentioned cells. gNO chamber allowed control and adjustment of following factors in all in vitro studies: gNO dose, total air flow, NO₂ levels, O₂ levels, CO₂ levels, temperature, and humidity.

Bacterial Study: Suspensions of Staphylococcus aureus and Pseudomonas aeroginosa cells were prepared in Tryptic Soy Broth medium, and then plated onto clear Tryptic Soy Agar (TSA) plates at various dilutions to achieve a countable range. A set of 4 cultured plates was incubated in each treatment and control exposure tubes inside the gNO chamber (FIG. 4) at 37° C. and relative humidity (RH) of 80% for period of 24 hours. Plates from the treated group were exposed to various concentrations of gNO (50, 80, 120, 160, and 200 ppm) mixed with medical air at a constant flow of about 5 L/min. Control plates within the chamber were exposed to only medical air at 5 L/min. Four cultured plates were placed inside conventional laboratory incubator at 37° C. with passive aeration for the duration of experiment. This served as a control for bacterial growth within the gNO exposure chamber. Following the incubation period, a count of colony forming units (CFU) was obtained. The difference in CFU between control and exposed plates were used to evaluate the bactericidal effects of nitric oxide. Results were analyzed using an unpaired student to test.

Fibroblast Study: fibroblast cells obtained from adult patients undergoing elective reconstructive surgery were cultured in Dulbeco's Modified Eagle's Medium (DMEM), supplemented with 10% fetal bovine serum (FBS) and antibiotic-antimycotic preparation and divided into ten 25 cm² vented culture flasks (COSTAR). Four of these flasks (treated group) were exposed to 20 or 200 ppm humidified gNO inside a specialized NO incubation chamber at 37° C. for 24 and 48 hours. The NO exposure chamber was validated prior to the study to eliminate extraneous variables and ensure optimal conditions for fibroblast cell growth. Another four flasks (control group) were placed inside conventional culture incubator and exposed only to ambient humidified air at 37° C. Two flasks were separately harvested and counted as the number of cells at zero time. Following the treatment, fibroblast cells were harvested and evaluated for morphology, cell count, capacity to proliferate and medium pH.

Animal study: In the animal model, full-thickness cutaneous wounds (Set A: 4 rabbits with Eight 8.0 mm punch biopsies & Set B: 4 rabbits with TWO 50×15 mm wounds) were made on each side of dorsal midline and infected with equal volume of Staphylococcus aureus suspension on DAY 0. On DAY 1, treated groups in A and B were respectively exposed to 200 and 400 ppm gNO for total of THREE days. Set A was exposed for TWO 4 hour sessions, interrupted by 1 hour of rest, inside a specialized restraining exposure chamber. A 24-hour continuous delivery model was used for animals in Set B by design of a specialized wound patch. Control groups were only exposed to medical grade air with corresponding flow rate. FOUR random sample punch biopsies (8.0 mm) were collected on post wounding days 3 and analyzed for bacterial content. Another FOUR punch biopsies from both wound and normal skin tissue were collected for fibroblast viability analysis and toxic effects of gNO.

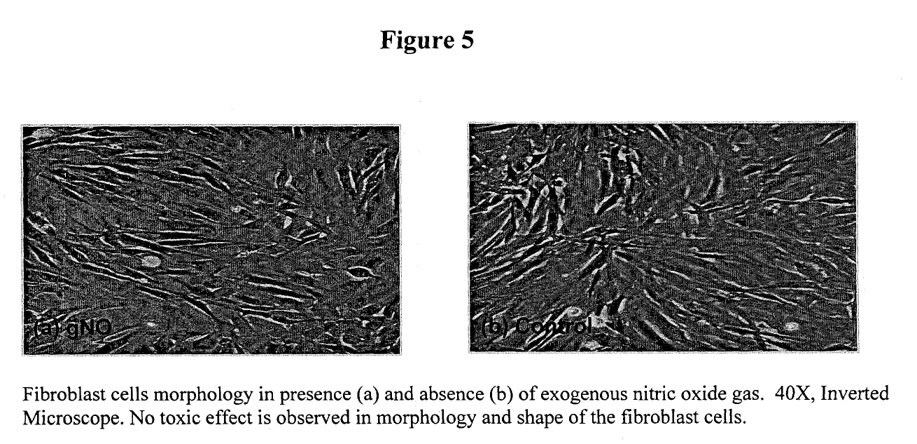

FIG. 5 shows morphology of fibroblast cells from the viability study, where cultured human fibroblast cells were exposed to various gNO concentrations less than 200 ppm continuously for 48 hours. Morphological appearance and attachment capacity of control and treated dermal fibroblasts cells following 48 hours period were quite comparable. Cells under gNO appeared healthy and attached to the culture plates. No toxic effect due to exposure to gNO was observed.

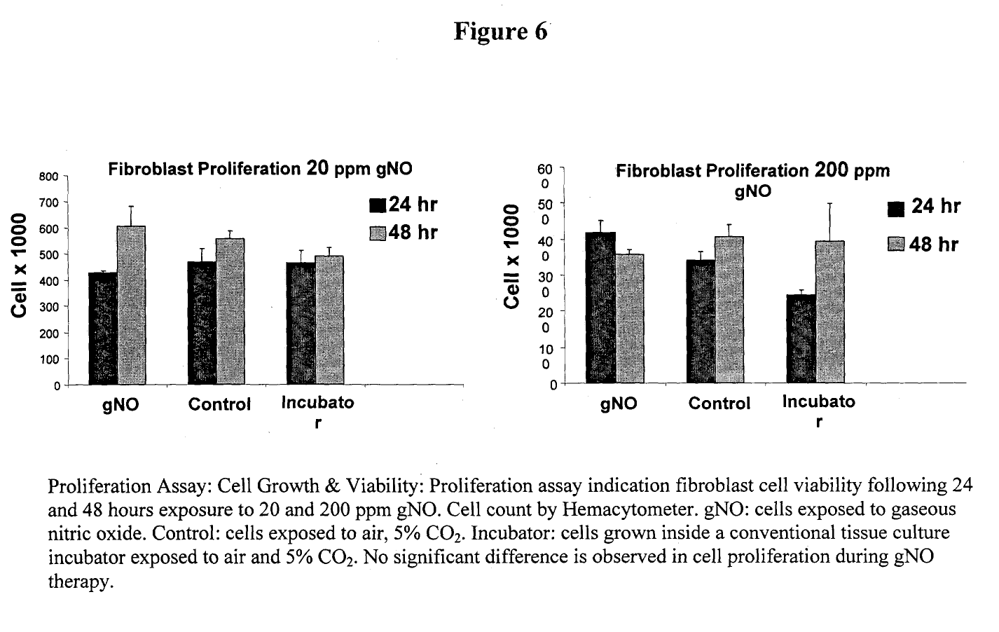

FIG. 6 reveals the results from the cell proliferation assay study. It compared the cellular growth between control and treated group exposed to 20 and 200 ppm gNO for 24 and 48 hours. Again, no significant variation in total cell count of dermal fibroblasts was observed between control and treated groups following 24 and 48 hours exposure to gNO (p<0.05).

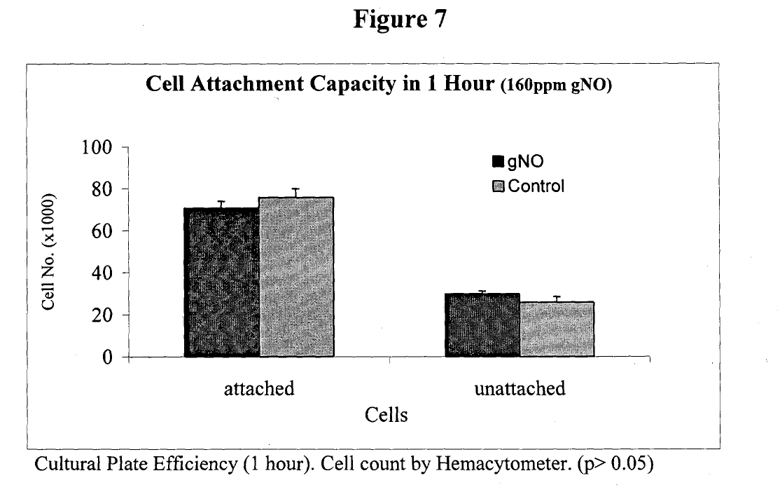

FIG. 7 shows results from Cell Attachment Capacity from the fibroblast cells exposed to 160 ppm of gNO. Capability of cells to reattach to the culture plates within a specified time limit is commonly used as an indication of viability of cells in culture. Both the control and treated groups show a 70% attachment capacity within 1 hour of culturing. This result in conjunction with cell morphology and count support the safety of gNO therapy for topical applications on mammalian skin tissue at least at a range between 100 to 200 ppm of gNO.

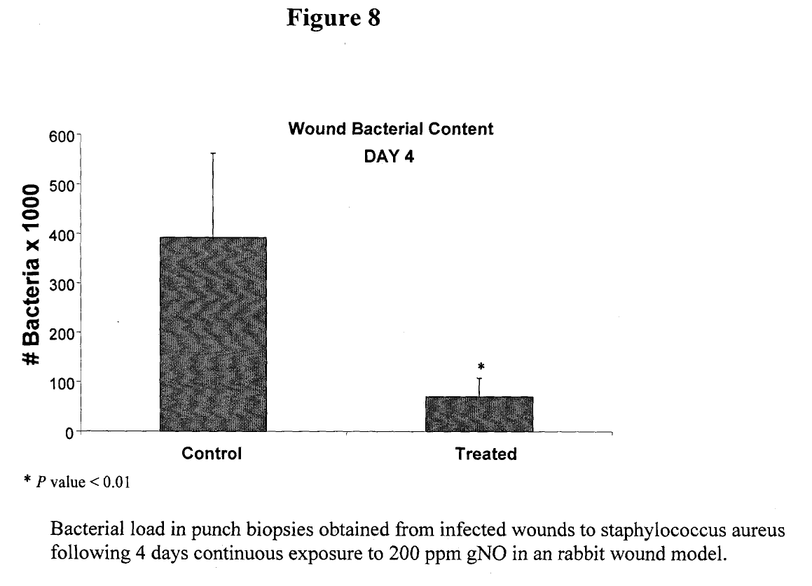

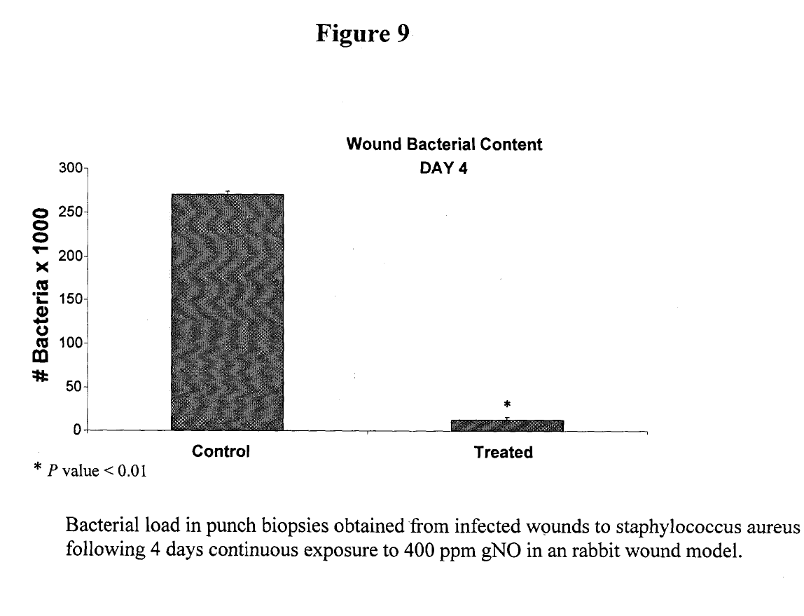

FIG. 8 reveals data from the animal study on bacterial content of the wounds exposed to 200 ppm gNO continuously for 72 hours when compared to control group only exposed to medical air. A significant bacterial reduction is observed in treated wounds. Rabbits appeared comfortable and at ease during the therapy and no toxic effect or damage were observed in the skin of treated animals when compared to the control. A similar device as shown in FIGS. 1 and 2 was used in this study. NO₂ did not exceed safety limits, at any point of the study, set by Occupational Safety and Health Administration (<4.3±0.3 ppm). FIG. 9 shows similar set of data as seen in FIG. 8, but where animal wounds were exposed to 400 ppm of gNO therapy. On average well over 10 fold drop (p<0.05) in bacterial content is observed in comparison between control and treated groups.

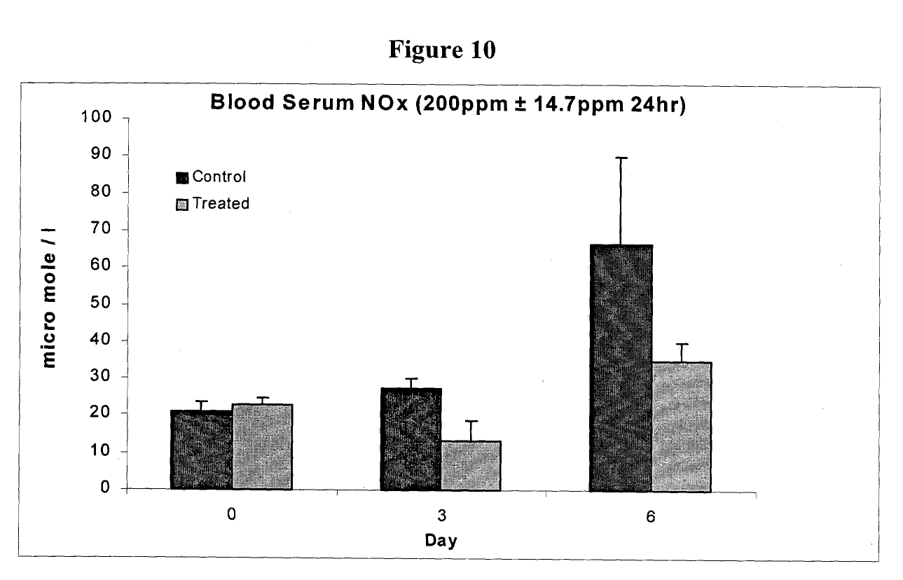

FIG. 10 demonstrates nitrogen oxides levels (NO₂ and NO₃), one of end products of nitric oxide metabolism, measured in blood serum collected from the animals following exposure to 200 ppm gNO intermittently for 6 days. None of the samples show an increased level of NOx due to exposure to gNO indicating the fact that exposing full thickness wounds (8 at 8.0 mm in diameter) will not increase the nitric oxide level in animal's circulation system.

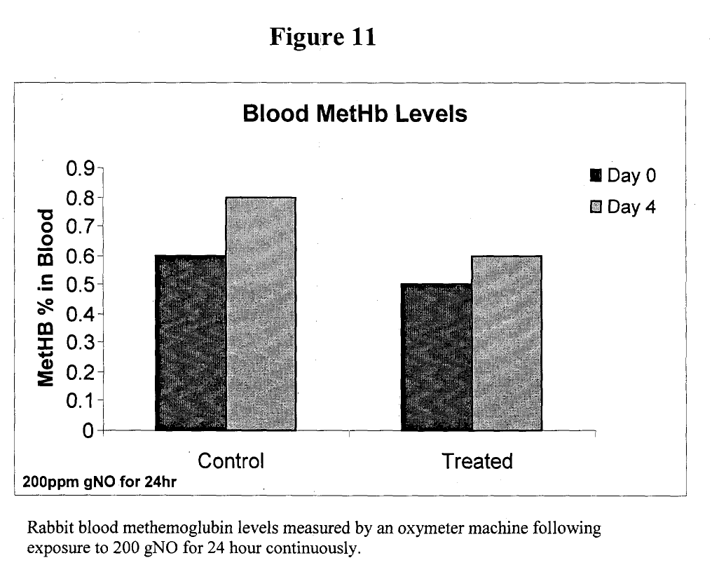

FIG. 11 indicates the level of methemoglobin (MetHb) in animal's blood following 6 day intermittent exposure to 200 ppm gNO. Animals in the treated group did not show an increase level of MetHB in comparison with the control group exposed to air. This further supports the data presented in FIG. 10 to the fact that topical application of gNO on open wounds did not contribute to an increase level of nitric oxide in the circulation.

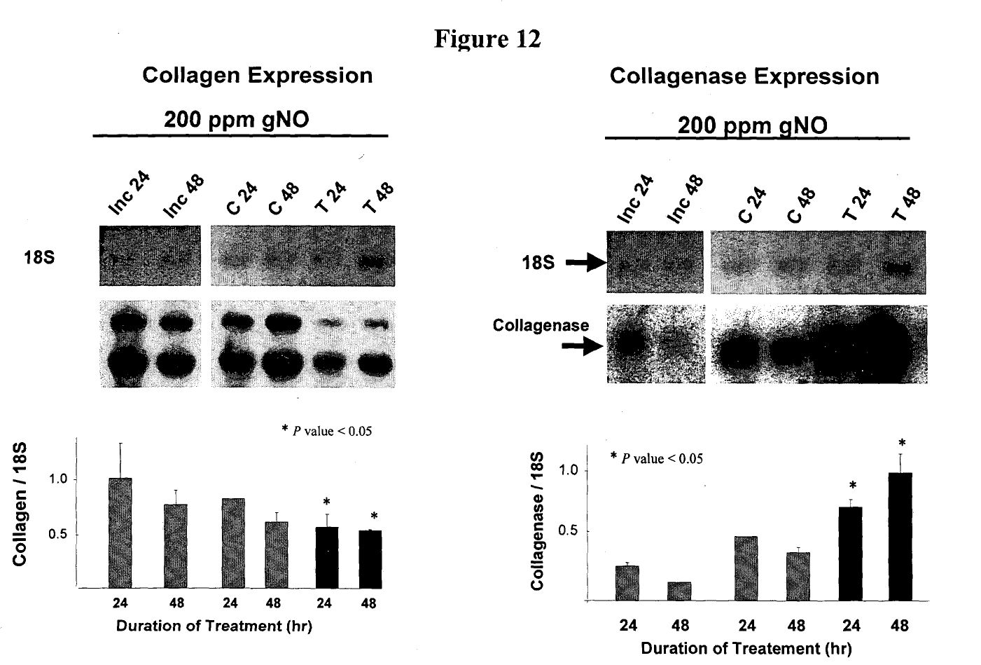

FIG. 12 presents mRNA expression of two important factors in wound healing process, namely collagen and collagenase, treated with high concentration of gNO (200 ppm). A drop in collagen activity is observed at this dose at both 24 and 48 hour exposure indicating a potential for gNO therapy in conditions where excessive healing is present (e.g. hypertrophic scarring). The analysis of collagenase expression further supports the fact that gNO at 200 ppm is not damaging the cellular function, as a significant increase in mRNA activity of this protein is observed.

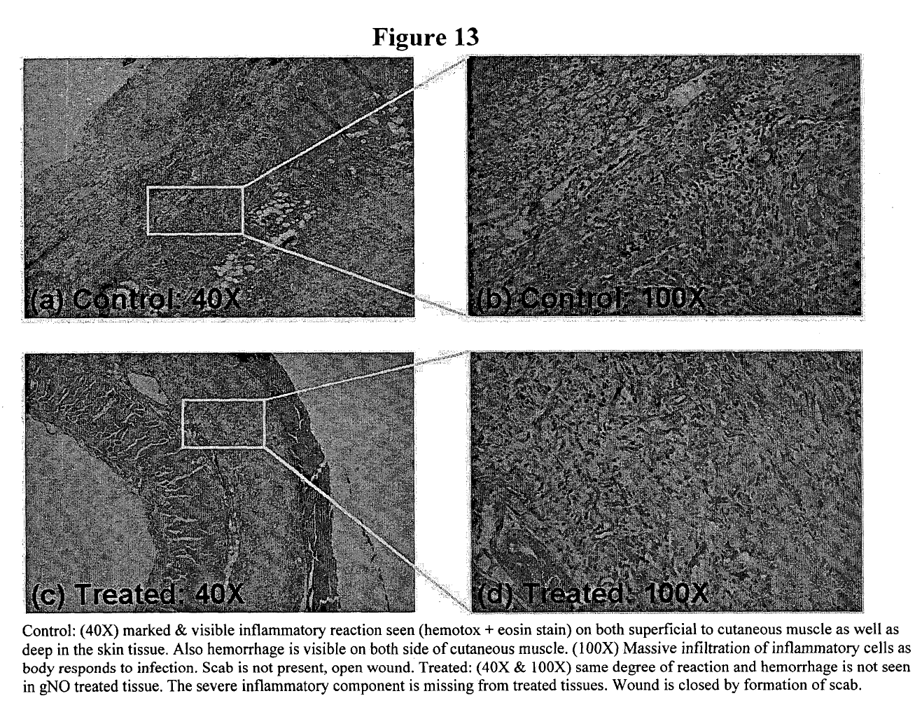

FIG. 13 presents histological analysis of tissue blocks prepared on wound punch biopsies from animals in treated and control groups. Samples from the control group show more advanced neutrophil infiltration and so a higher degree of inflammatory reaction. A lower level of neutrophil concentration is seen in wounds treated with gNO. Wounds treated with gNO show a layer of scab closing on the wound, but control wounds remain open for longer period of time. Overall, a healthier healing process is observed in the wounds treated with gNO. No toxic effects (cellular debris due to apoptosis) can be seen in gNO treated group.

In addition, the role of NO in the survival of tissue (skin) flap has been extremely beneficial. In a free flap, the flap tissue is completely removed from the donor site and attached to the wound by micro vascular techniques. In this case there will be a base that provided circulatory support for the flap.

Nitric oxide synthesized by vascular endothelium is responsible in regulation of vascular tone. Through this action, nitric oxide relaxes vascular tone and increases local blood flow protecting against ischemia-induced flap necrosis.

In flap surgery reperfusion to ischemic tissue and organs is an essential criterion in survival of the tissue. In many surgical procedures this step can lead to intensified tissue injury caused by reperfusion edema. Therefore, administration of exogenous gNO can potentially maintain the vascular tone and protect endothelium cells from the ischemia/reperfusion injury.

Having described preferred embodiments of the invention with reference to the drawings and graphs, it is to be understood that the invention is not limited to these precise embodiments, and that various changes and modifications may be effected therein by one skilled in the art without departing from the scope of the invention as defined in the appended claims.

What is claimed is:

1. A method for promoting the healing of damaged tissue in a patient in need of such treatment, comprising:
exposing the damaged tissue, which is surrounded by an air impermeable wound cover, to an effective amount of gaseous nitric oxide for a period of time that exceeds eight consecutive hours; and
allowing at least a portion of the gaseous nitric oxide to contact the air adjacent to the air impermeable wound cover through the air impermeable wound cover.

2. The method of claim 1 wherein the damaged tissue is selected from the group consisting of muscle, ligament, tendon, skin, and bone.

3. The method of claim 1 wherein the damaged tissue is damaged by surgical incisions, trauma, and pathological processes.

4. The method of claim 1 further comprising healing the damaged tissue by recruiting inflammatory cells, followed by fibroblasts to the damaged tissue.

5. The method of claim 1 wherein the air impermeable wound cover is transparent and allows for permeation of small molecules, while simultaneously preventing microbial invasion.

6. The method of claim 1 wherein effective amount of gaseous nitric oxide ranges from 20-1000 ppm.

7. The method of claim 1 further comprising pretreatment step of cleaning and scrubbing the damaged tissue.

8. The method of claim 1 further comprising pretreatment step of exposing to a wound healing agent other than gaseous nitric oxide.

9. The method of claim 1 further comprising pretreatment step of exposing the damaged tissue to an agent in combination with gaseous nitric oxide in order to enhance its effectiveness and/or absorption.

10. The method of claim 1 further comprising posttreatment step of wetting, dampening or moistening the damaged tissue following gaseous nitric oxide therapy.

11. The method of claim 1 further comprising posttreatment step of applying a wound healing agent in combination to gaseous nitric oxide therapy.

12. The method of claim 1 further comprising posttreatment step of exposing the damaged tissue to an agent in combination with gaseous nitric oxide in order to enhance its effectiveness and/or absorption.

13. The method of claim 1 further comprising the administration of exogenous nitric oxide gas to tissue flap and surrounding damaged area in order to promote flap viability and increase local blood flow to donated tissue.

14. A method for promoting the healing of damaged tissue in a patient in need of such treatment, comprising:
spraying, from a spray container, the damaged tissue with an effective amount of gaseous nitric oxide; and
allowing the gaseous nitric oxide to contact the air adjacent the damaged tissue.

15. A method for promoting the healing of damaged tissue in a patient in need of such treatment, comprising:
exposing the damaged tissue, which is surrounded by an air impermeable wound cover, to an effective amount of gaseous nitric oxide for a period of time that exceeds eight consecutive hours.

16. A method for promoting the healing of damaged tissue in a patient in need of such treatment, comprising:
exposing the damaged tissue, which is surrounded by an air impermeable wound cover, to an effective amount of gaseous nitric oxide and other gases which contain oxygen for a period of time.
